(19) United States
(12) Patent Application Publication
(10) Pub. No.: US 2023/0392105 A1
(43) Pub. Date: Dec. 7, 2023

(54) BIOREACTOR MEDIA CONDITION SYSTEM AND RELATED METHODS

(71) Applicant: CORNING INCORPORATED, Corning, NY (US)

(72) Inventor: Stephen Joseph Caracci, Elmira, NY (US)

(21) Appl. No.: 18/032,042

(22) PCT Filed: Oct. 27, 2021
(86) PCT No.: PCT/US2021/056735

§ 371 Date: Apr. 14, 2023

Related U.S. Application Data
(60) Provisional application No. 63/107,871, filed on Oct. 30, 2020.

(51) Int. Cl. C12M 1/00; C12M 1/04; C12M 1/34

(52) U.S. Cl. CPC C12M 29/26; C12M 23/28; C12M 23/24; C12M 23/34; C12M 29/06; C12M 29/10; C12M 41/12; C12M 41/46

(57) ABSTRACT

A cell culture system is provided that includes a cell culture vessel enclosing an interior configured to culture cells in liquid cell culture media, the cell culture vessel comprising a bioreactor inlet for supply of cell culture media to the interior and a bioreactor outlet for removal of cell culture media from the interior; and a media conditioning system comprising an enclosure enclosing an interior space and a gas exchange system, the gas exchange system comprising a media vessel, wherein the media vessel comprises at least one media cavity configured to contain cell culture media and a gas permeable membrane separating the media cavity from the interior space.

### CROSS-REFERENCE TO RELATED APPLICATIONS

This application claims the benefit of priority under 35 U.S.C. § 119 of U.S. Provisional Application Ser. No. 63/107,871 filed on Oct. 30, 2020, the content of which is relied upon and incorporated herein by reference in its entirety.

### FIELD OF THE DISCLOSURE

This disclosure generally relates to media conditioning systems using gas permeable films, and bioreactors and bioreactor systems for cell culture with media conditioning systems with gas permeable films.

### BACKGROUND

In the bioprocessing industry, large-scale cultivation of cells is performed for purposes of the production of hormones, enzymes, antibodies, vaccines, and cell therapies. Cell and gene therapy markets are growing rapidly, with promising treatments moving into clinical trials and quickly toward commercialization. However, one cell therapy dose can require billions of cells or trillions of viruses. As such, being able to provide a large quantity of cell products in a short amount of time is critical for clinical success.

A significant portion of the cells used in bioprocessing are anchorage dependent, meaning the cells need a surface to adhere to for growth and functioning. Traditionally, the culturing of adherent cells is performed on two-dimensional (2D) cell-adherent surfaces incorporated in one of a number of vessel formats, such as T-flasks, petri dishes, cell factories, cell stack vessels, roller bottles, and HYPERStack® vessels. These approaches can have significant drawbacks, including the difficulty in achieving cellular density high enough to make it feasible for large scale production of therapies or cells. Alternative methods have been suggested to increase volumetric density of cultured cells. These include microcarrier cultures performed in stir tanks. Another example of a high-density cell culture system is a hollow fiber bioreactor, in which cells may form large three-dimensional aggregates as they proliferate in the interspatial fiber space.

Yet another example of a high-density culture system for anchorage dependent cells is a packed-bed bioreactor system. In this this type of bioreactor, a cell substrate is used to provide a surface for the attachment of adherent cells. Medium is perfused along the surface or through the semi-porous substrate to provide nutrients and oxygen needed for the cell growth. For example, packed bed bioreactor systems that contain a packed bed of support or matrix systems to entrap the cells have been previously disclosed U.S. Pat. Nos. 4,833,083; 5,501,971; and 5,510,262. Packed bed matrices usually are made of porous particles as substrates or non-woven microfibers of polymer. Such bioreactors function as recirculation flow-through bioreactors.

In these various types of cell culture reactors, the cells must be grown under controlled conditions, including being suspended or perfused in a cell culture media, which is a liquid media containing nutrients necessary for cell life and growth. The contents of the cell culture media, including the pH and content of dissolved gases, as well as the temperature of the media and/or the cells, must be controlled to optimize cell growth and performance of the bioreactor. Therefore, media conditioning systems are used in combination with or integrated into the bioreactors to condition the media therein. These media conditioning systems can, for example, control temperature, pH, carbon dioxide content, dissolved oxygen content, and other aspects of the media. Depending on the cells being grown or the stage of the culture process, the specific media conditioning needs can vary. Typically, media conditioning may be performed in a hollow container or vessel (e.g., a beaker or bottle) with a complicated system of probes to control the composition of the media, one or more stirrers to mix the media, and some type of temperature control jacket around the vessel. The probes may enter the vessel through a cap on the vessel body, and sterility is always a concern, especially if the system is open or opened during use.

However, there is a need for improved media conditioning systems that can meet the demands of scalable cell culturing platforms, while providing a simple, reliable, and closed architecture.

### SUMMARY

According to an embodiment of this disclosure, a cell culture system is provided that includes a cell culture vessel enclosing an interior configured to culture cells in liquid cell culture media, the cell culture vessel comprising a bioreactor inlet for supply of cell culture media to the interior and a bioreactor outlet for removal of cell culture media from the interior; and a media conditioning system comprising an enclosure enclosing an interior space and a gas exchange system, the gas exchange system comprising a media vessel, wherein the media vessel comprises at least one media cavity configured to contain cell culture media and a gas permeable membrane separating the media cavity from the interior space

### BRIEF DESCRIPTION OF THE DRAWINGS

FIG. 1 shows a bioreactor system with a media conditioning vessel known in the art.

FIG. 2 shows a schematic of a cell culture system according to one or more embodiments of this disclosure.

FIG. 3 shows a media conditioning system according to one or more embodiments of this disclosure.

FIG. 4 shows a close up view of a section of the media conditioning system indicated by the dotted outline in FIG. 3.

FIG. 5 shows a media conditioning vessel according to one or more embodiments of this disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not limiting and merely set forth some of the many possible embodiments of the claimed invention.

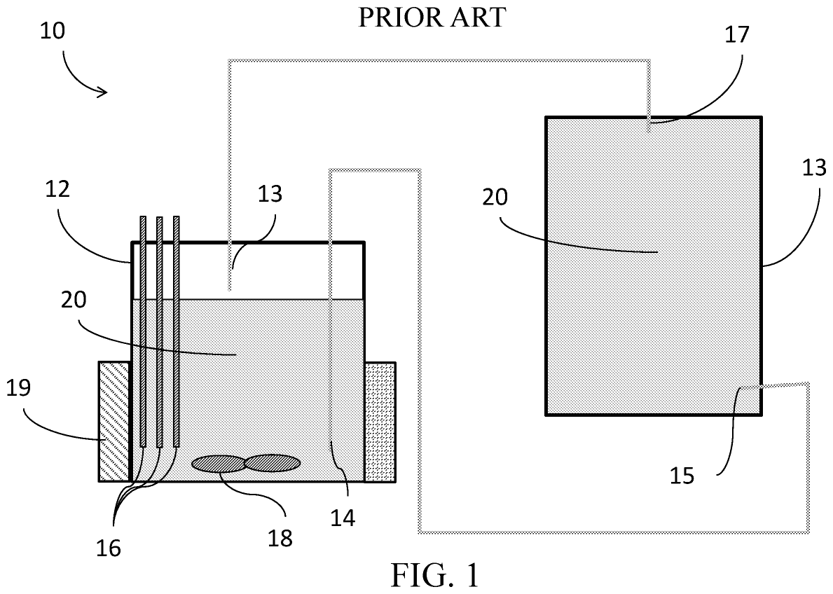

FIG. 1 shows a cell culture system 10 with a conventional media conditioning vessel 12 attached to a bioreactor vessel 13. Media 20 flows out of the media conditioning vessel 12 via the outlet 14 to an inlet 15 of the bioreactor vessel 13. The media 20 within the bioreactor vessel 13 is used to feed cells (not shown) within the bioreactor vessel 13, and may be perfused therethrough to an outlet 17. The media 20 can then, for example, be recirculated to the media conditioning vessel 12 via inlet 13. The media conditioning vessel 12 may be a simple hollow container or vessel (e.g., a glass or plastic beaker or bottle). A complicated system of probes 16 are used to control the composition of the media 20, and one or more stirrers 18 are used to mix the media 20. A temperature control jacket 19 (e.g., water jacket) is shown around the media conditioning vessel 12, which can be used to control temperature of the media 20. The probes 16 can include a combination of sensors and feeds for various components (e.g., oxygen) and may enter the media conditioning vessel 12 through a cap on the vessel body. The complexity of this design and the various parts involved increases risks of contamination and complicates the setup and/or disposal of the system. As discussed below, embodiments of this disclosure provide a simplified and elegant media conditioning system.

Embodiments of this disclosure are directed to media conditioning vessels and systems, and cell culture systems incorporated media conditioning systems. In particular, the media conditioning systems described herein use a gas-permeable membrane to condition the media before it is supplied to or perfused through a bioreactor. In some embodiments, it is contemplated that the bioreactor is a fixed bed or packed bed bioreactor with a high density scaffold for cell growth. The system also allows for temperature conditioning of the media and for control of other aspects, such as pH.

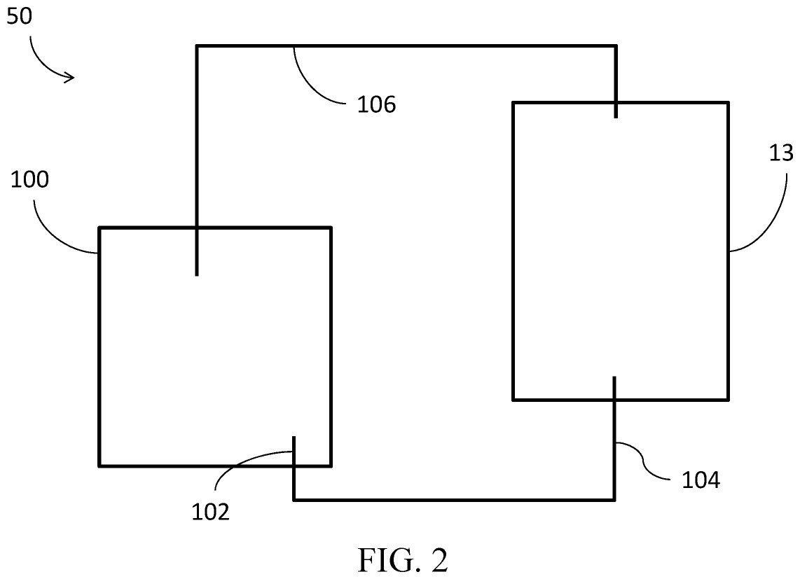

FIG. 2 shows a schematic of a cell culture system 50, according to some embodiments, that includes both a bioreactor vessel 13 for growing cells and/or viral vectors, and a media conditioning system 100 to condition media that is supplied to the bioreactor vessel. As shown in FIG. 2, the system 50 can be configured in a recirculation loop, where media flows out of the media conditioning system 100 via on outlet 102 to the bioreactor vessel 13 via tubing 104 or other fluid connector. Within the bioreactor vessel 13, the media supplies necessary nutrients to the cells and maintains a healthy cell environment. Optionally, the media can be returned to the media conditioning vessel 100 via a return tubing 105 or other connector. Embodiments of this disclosure include the entire cell culture system 50, as well as the individual media conditioning system 100, which can be used to condition media in a variety of systems or applications.

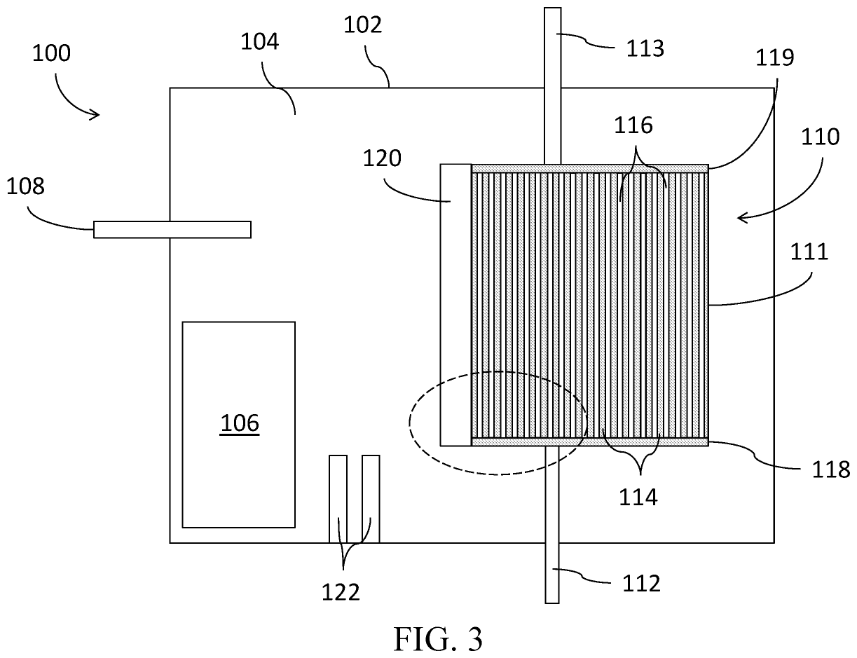

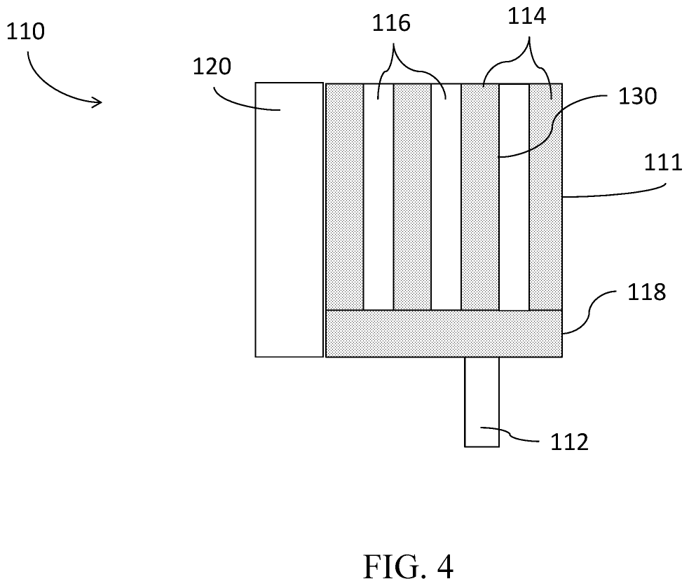

FIG. 3 shows a schematic of the media conditioning system 100, according to some embodiments. The media conditioning system 100 includes an enclosure 102 that encloses an interior space 104 configured to be supplied by one or more gases via a gas supply 108, and a gas exchange system 110 within the interior space 104. The gas exchange system 110 includes a media vessel 111 having at least one media cavity 114 configured to contain cell culture media, where the media vessel 111 includes a gas permeable surface 130 (see FIG. 4) separating the media cavity 114 from a tracheal space 116 contain gas from the gas supply 108. The gas permeable surface 130 is configured to allow gas from the tracheal space 116 or interior space 104 to pass through the gas permeable surface 130 to media within the media cavity 114. It should be appreciated that FIG. 3 is not necessarily drawn to scale.

In the case of multiple media cavities 114, a manifold 118 can distribute media from a media inlet 112 to the multiple media cavities 114. Likewise, a manifold 119 can collect media from the multiple media cavities 114 and channel it to a media outlet 113, from where the media is then carried to the bioreactor or vessel requiring the media. Similarly, when having multiple media cavities 114, the media vessel 111 can have multiple tracheal spaces 116, where the tracheal spaces 116 and media cavities 114 are arranged in an alternating manner. These tracheal spaces 116 can also be connected to one another via a gas manifold 120 that supplies the gas to all of the tracheal spaces. The gas manifold 120 can further be equipped with a gas circulator (not shown) to force the gas through the tracheal spaces 116 at a desired rate, depending on the conditioning needs and media flow rate of the media conditioning system 100.

FIG. 4 shows a close up view of the media vessel 111 in the region indicated by the dotted line in FIG. 3. FIG. 4 is not necessarily drawn to scale. It is contemplated that the media cavities 114 can have one or multiple sides that have a gas permeable membrane 130. Further details of this constructions are discussed below. In some embodiments, the multiple media cavities 114 with interstitial tracheal spaces 116 is provided as a single integral device. Alternatively, the media cavities 114 can be stackable subunits, which stack in such a way so as to leave tracheal spaces 116 between the individual cavities.

Optionally, the media conditioning system 100 is temperature controlled. That is, the temperature of the space 104 within the enclosure 102 can be heated or cooled control the temperature of gas and/or media that passes into or through the gas exchange system 110. In some embodiments, the temperature control is used to heat or cool the gas, which then heats or cools the media after the gas exchange occurs through the gas permeable surface 130. Optionally, this temperature control can be achieved by an integrated temperature control device 106 in the enclosure 102, such as, for example, a heater or cooler. Alternatively, the gas can be temperature controlled prior to entering the enclosure 102.

The media conditioning system 100 can also include one or more sensors 122 for detecting qualities of the gas and/or media. In FIG. 3, the sensors 122 are provided in the enclosure for measuring properties of the gas. However, sensors can also be supplied in line with the media, either up or down stream of the media inlet 112 and media outlet 113, or can be supplied in the bioreactor vessel itself. Based on feedback from the sensors 112, the components of the gas via inlet 108 and/or the temperature control device 106 can be controlled to achieve the desired composition and temperature of the cell culture media for optimal performance of the culture. For example, gas composition can be adjusted to achieve a desired O₂ and/or CO₂ level; and pH can be adjusted.

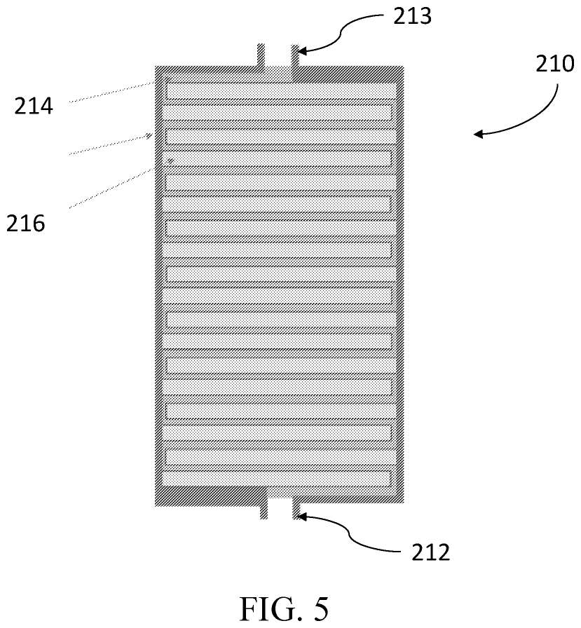

FIG. 5 shows an alternative embodiment of a media conditioning vessel 210, according to one or more embodiments. The functionality is similar to that discussed above in FIG. 2-4, but instead of multiple media cavities that are manifolded together, the media conditioning vessel 210 has media cavity 214 that follows a long, circuitous path through and around multiple tracheal spaces 216 as media flows from the media inlet 212 to the media outlet 213.

The enclosure 102 can be a thermal enclosure. As used herein, “thermal enclosure” means that the enclosure is thermally insulated so a temperature in the interior of the thermal enclosure can be more easily controlled. As an alternative or in addition to a thermal enclosure, the enclosure 102 can include a heat source (106) for controlling a temperature within the enclosure, or a temperature of a gas within the enclosure, as discussed above. The heat source may be an integral structure and function of the enclosure 102, or it could be a separate component that the enclosure 102 is configured to accept, as needed. According to some embodiments, the heat source provides heat to the gas that is sufficient to control a temperature of the media in the media exchange system within a desired range for cell culture.

In some embodiments, the media conditioning system further includes one or more sensors for sensing a property of the gas within the enclosure, or of the media within the gas exchange system, or of the media prior to entering, after exiting, or while within the bioreactor. The one or more sensors can measure temperature, pH, oxygen (O₂), CO₂, or any of a number of variables that are relevant to the cell culture operation being performed.

The gas exchange system has an inlet and an outlet connected to the media cavity, so that media can flow into the media cavity of the gas exchange system, and can then flow across one or more gas permeable surfaces on its way to the outlet. The outlet is then fluidly connected to a bioreactor to feed the cell culture media to a space for cell culture. In some embodiments, the gas exchange system can include one or more tracheal spaces separating a plurality of media cavities. In an aspect of some embodiments, the media cavity follows a circuitous path through the gas exchange system to maximize the surface area of media exposed to the gas permeable surface.

There are many advantages to the media conditioning systems and vessels disclosed herein. For example, the design allows the media conditioning vessel to be a single-use or disposable vessel. The reusable or single use components of the system can reduce cost of operation, while achieving gas transport to the media through the gas permeable membrane in a closed system. The gas can also be temperature controlled in the system, which thus effectively controls the temperature of the media. In addition, due to the possible embodiments, the system can handle large amounts of media, and do so efficient. Further, the simplified design can avoid the use of adhesives or high-particulate materials, thus avoiding potential complications or undesired components in the bioprocessing industry.

Further details of embodiments of the media conditioning vessel are now provided. According to an illustrative embodiment, the media cavity 114 includes a unitary body including a bottom tray and a top plate, connected by side walls and end walls. At least one aperture located along any periphery of the apparatus permits access to the internal volume. At least one gas permeable substrate/membrane 130 is affixed to a support internal to the body of the media cavity 114. A tracheal space 116 permits gases from the space 104 or gas manifold 120 to be exchanged across the gas permeable, liquid impermeable membrane, into and out of the media cavity 114. Further, the tracheal space 116 is an air chamber confined by the outer enclosure 102. Communication between a tracheal space 116 and the media within the media cavity 114 provides a uniform and efficient gas exchange along the length of the media cavity 114.

One embodiment of the media vessel 111 includes a plurality of media cavities 114, each having a gas permeable membrane 130 that is liquid impermeable and an opposing surface. At least one tracheal space 116 is in communication with at least one gas permeable membrane 130 so that media within the media cavities 114 can exchange gases (e.g. oxygen, carbon dioxide, etc.) with the space 104 of the enclosure. The media vessel 111 can have at least one tracheal space 116 incorporated with a plurality of media cavities 114 combined into one integral unit. The integral unit thus has multiple media flow pathways through the multiple media cavities 114 in any assembled arrangement. A preferred embodiment of a media vessel 111 alternates each media cavity 114 with a tracheal space 116 in a vertical successive orientation whereby each media cavity includes a substantially planar horizontal surface along which media can flow. The media cavity 114, however, may be planar and/or nonplanar to accommodate different geometries or increase the surface area available for gas exchange. A modified or enhanced surface area in combination with one or more tracheal spaces 116 enables a diversified area for gas exchange with the media.

When a plurality of media cavities 114 are arranged with tracheal spaces 116 formed there-between, the tracheal spaces 116 permit gaseous exchange between the gas permeable (liquid impermeable) membrane 130 and the space 104 of the enclosure 102 or gas manifold 120. In a preferred embodiment, each media cavity 114 alternates with a tracheal space 116 allowing the cells greater access to external gaseous exchange.

One embodiment of the media conditioning system 110 uses a gas permeable (liquid impermeable) membrane 130 as the opposing surface of a media cavity 114. In such an embodiment, a plurality of gas permeable substrates 130 (internal to the body of the apparatus) can be incorporated to increase surface area for gas exchange with the media. Each gas permeable substrate 130 may have a tracheal space 116 above and/or below it. One such embodiment is capable of incorporating one or more tracheal spaces 116 between each stacked gas permeable substrate 130.

Preferably, the tracheal space(s) 116 provide uniform gaseous distribution within the media vessel 111. Completely filling the vessel 111 with media would allow for optimal gas or cellular nutrient exchange. The plurality of layers of gas permeable substrates are further capable of being interconnected or adjoined to provide a multiplicity of areas for media exchange. The plurality of modules may be interconnected in series or staggered to permit continuous flow. For easy assembly and disassembly, individual units having snap-like features could be securely and easily adjoined. In an aspect of some embodiments, the apparatus comprises a manifold to access the media cavities 114 of an integral unit. The manifold 118/119 may further be capable of directing the flow of air, liquid, media and/or cellular material within the media cavity 114.

For the addition and removal of media, the media conditioning system 110 has at least one media inlet 112 and media outlet 113. Each media cavity 114, however, may have individual inlets/outlets. Supplementary, the apparatus is capable of being equipped with a septum seal accessible opening or aperture either integrated within the body of the apparatus itself, or as a part of a cap. When a cap is utilized, in an embodiment having a height as measured by the distance between an outermost plane of the bottom tray and an outermost plane of the top plate, has a cap, cover, and/or septum covering the inlet or outlet. The cap may have a diameter that does not exceed the height of the vessel so as to prevent interference when the multiple vessels are stacked. Additionally, the cap may be integrally included in a top surface, side, and/or corner region of the apparatus. When the media cavities 114 having the gas permeable membranes 130 are stacked, the inlets and outlets may be positioned in a parallel or staggered assembly so as to permit flow or perfusion of media through the cavities.

The media conditioning vessel 111 of the present invention may be made by any number of acceptable manufacturing methods well known to those of skill in the art. In a preferred method, the media conditioning vessel 111 is assembled from a collection of separately injection molded parts. Though any polymer suitable for molding and commonly utilized in the manufacture of laboratory ware may be used, polystyrene is preferred. Although not required, for optical clarity, it is advantageous to maintain a thickness of no greater than 2 mm.

Gas permeable, liquid impermeable membrane 130 or substrates may be comprised of one or more membranes known in the art. Membranes typically comprise suitable materials that may include for example: polystyrene, polyethylene, polycarbonate, polyolefin, ethylene vinyl acetate, polypropylene, polysulfone, polytetrafluoroethylene (PTFE) or compatible fluoropolymer, a silicone rubber or copolymer, poly(styrene-butadiene-styrene) or combinations of these materials. Various polymeric materials may be utilized. For its known competency, then, polystyrene may be a preferred material for the membrane (of about 0.003 inches in thickness, though various thicknesses are also permissive of cell growth). As such, the membrane may be of any thickness, preferably between about 25 and 250 microns, but ideally between approximately and 125 microns. The membrane allows for the free exchange of gases between the interior space of the enclosure and the media cavity and may take any size or shape, so long as the membrane is supportive of media flow and gas exchange. A preferred embodiment would include a membrane that is additionally durable for manufacture, handling, and manipulation of the apparatus.

The gas permeable membrane 130 is properly affixed to the supports in the media conditioning vessel 111 by any number of methods including but not limited to adhesive or solvent bonding, heat sealing or welding, compression, ultrasonic welding, laser welding and/or any other method commonly used for generating seals between parts. Laser welding around the circumference of the membrane 130 is preferred to establish a hermetic seal around the membrane region such that the membrane is flush with and fused to the face of the supports such it becomes an integral portion of the interior surface of the vessel 111. Once the gas permeable membrane 130 is adhered, then the top plate and bottom tray may be joined. The parts are held together and are adhesive bonded along the seam, ultrasonically welded, or laser welded. Preferably, laser welding equipment is utilized in a partially or fully automated assembly system. The top plate and tray are properly aligned while a laser weld is made along the outer periphery of the joint.

Optionally, the interior surfaces of the media cavities and/or gas permeable membranes may be treated for sterility and compatibility with cell culture media. Treatment may be accomplished by any number of methods known in the art which include plasma discharge, corona discharge, gas plasma discharge, ion bombardment, ionizing radiation, and high intensity UV light.

For easier accessibility and manufacturing of the multilayered media conditioning vessel 111, the arrangement of media cavities 114 and tracheal spaces 116 into individual modular units may be preferred. For example, in a modular unit of one embodiment, an individual modular unit can include a support network in combination with gas permeable membranes. A plurality of such modular units is capable of being interconnected and/or interlocked or adhered together to provide a multiplicity of media cavities that can be easily assembled or disassembled into a unitary multilayered vessel for media conditioning. Vertical stacking of the modular units would be analogous to interconnecting building blocks. Any number of media cavities could be assembled or disassembled to provide a wide range of accessibility options to each modular unit. One embodiment uses supports forming a shelf/frame along a periphery of the individual unit in addition to lateral ribs spanning or bisecting the distance internal to the frame. The transparent gas permeable substrate(s) can be adhered to supports such that air gaps or tracheal spaces are formed between each media cavity to allow gas distribution throughout the unitary apparatus when multiple trays are assembled into one vessel body. The tracheal spaces have gaseous exchange with the external atmosphere of the enclosure. Further, the tracheal spaces provide air/gas exchange with media in the media cavities.

It is contemplated that the cell culture system 50 may be used with a bioreactor 13 having a packed-bed of fixed-bed cell culture substrate. In conventional large-scale cell culture bioreactors, different types of packed bed bioreactors have been used. Usually these packed beds contain porous matrices to retain adherent or suspension cells, and to support growth and proliferation. Packed-bed matrices provide high surface area to volume ratios, so cell density can be higher than in the other systems. However, the packed bed often functions as a depth filter, where cells are physically trapped or entangled in fibers of the matrix. Thus, because of linear flow of the cell inoculum through the packed bed, cells are subject to heterogeneous distribution inside the packed-bed, leading to variations in cell density through the depth or width of the packed bed. For example, cell density may be higher at the inlet region of a bioreactor and significantly lower nearer to the outlet of the bioreactor. This non-uniform distribution of the cells inside of the packed-bed significantly hinders scalability and predictability of such bioreactors in bioprocess manufacturing, and can even lead to reduced efficiency in terms of growth of cells or viral vector production per unit surface area or volume of the packed bed.

Another problem encountered in packed bed bioreactors disclosed in prior art is the channeling effect. Due to random nature of packed nonwoven fibers, the local fiber density at any given cross section of the packed bed is not uniform. Media flows quickly in the regions with low fiber density (high bed permeability) and much slower in the regions of high fiber density (lower bed permeability). The resulting non-uniform media perfusion across the packed bed creates the channeling effect, which manifests itself as significant nutrient and metabolite gradients that negatively impact overall cell culture and bioreactor performance. Cells located in the regions of low media perfusion will starve and very often die from the lack of nutrients or metabolite poisoning. Cell harvesting is yet another problem encountered when bioreactors packed with non-woven fibrous scaffolds are used. Due to packed-bed functions as depth filter, cells that are released at the end of cell culture process are entrapped inside the packed bed, and cell recovery is very low. This significantly limits utilization of such bioreactors in bioprocesses where live cells are the products. Thus, the non-uniformity leads to areas with different exposure to flow and shear, effectively reducing the usable cell culture area, causing non-uniform culture, and interfering with transfection efficiency and cell release.

To address these and other problems of existing cell culture solutions, embodiments of the present disclosure provide cell growth substrates, matrices of such substrates, and/or packed-bed systems using such substrates that enable efficient and high-yield cell culturing for anchorage-dependent cells and production of cell products (e.g., proteins, antibodies, viral particles). Embodiments include a porous cell-culture matrix made from an ordered and regular array of porous substrate material that enables uniform cell seeding and media/nutrient perfusion, as well as efficient cell harvesting. Embodiments also enable scalable cell-culture solutions with substrates and bioreactors capable of seeding and growing cells and/or harvesting cell products from a process development scale to a full production size scale, without sacrificing the uniform performance of the embodiments. For example, in some embodiments, a bioreactor can be easily scaled from process development scale to product scale with comparable viral genome per unit surface area of substrate (VG/cm²) across the production scale. The harvestability and scalability of the embodiments herein enable their use in efficient seed trains for growing cell populations at multiple scales on the same cell substrate. In addition, the embodiments herein provide a cell culture matrix having a high surface area that, in combination with the other features described, enables a high yield cell culture solution. In some embodiments, for example, the cell culture substrate and/or bioreactors discussed herein can produce 10¹⁶ to 10¹⁸ viral genomes (VG) per batch.

In one embodiment, a matrix is provided with a structurally defined surface area for adherent cells to attach and proliferate that has good mechanical strength and forms a highly uniform multiplicity of interconnected fluidic networks when assembled in a packed bed or other bioreactor. In particular embodiments, a mechanically stable, non-degradable woven mesh can be used as the substrate to support adherent cell production. The cell culture matrix disclosed herein supports attachment and proliferation of anchorage dependent cells in a high volumetric density format. Uniform cell seeding of such a matrix is achievable, as well as efficient harvesting of cells or other products of the bioreactor. In addition, the embodiments of this disclosure support cell culturing to provide uniform cell distribution during the inoculation step and achieve a confluent monolayer or multilayer of adherent cells on the disclosed matrix, and can avoid formation of large and/or uncontrollable 3D cellular aggregates with limited nutrient diffusion and increased metabolite concentrations. Thus, the matrix eliminates diffusional limitations during operation of the bioreactor. In addition, the matrix enables easy and efficient cell harvest from the bioreactor. The structurally defined matrix of one or more embodiments enables complete cell recovery and consistent cell harvesting from the packed bed of the bioreactor.

According to some embodiments, a method of cell culturing is also provided using bioreactors with the matrix for bioprocessing production of therapeutic proteins, antibodies, viral vaccines, or viral vectors.

In contrast to existing cell culture substrates used in cell culture bioreactors (i.e., non-woven substrates of randomly ordered fibers), embodiments of this disclosure include a cell culture substrate having a defined and ordered structure. The defined and order structure allows for consistent and predictable cell culture results. In addition, the substrate has an open porous structure that prevents cell entrapment and enables uniform flow through the packed bed. This construction enables improved cell seeding, nutrient delivery, cell growth, and cell harvesting. According to one or more particular embodiments, the matrix is formed with a substrate material having a thin, sheet-like construction having first and second sides separated by a relatively small thickness, such that the thickness of the sheet is small relative to the width and/or length of the first and second sides of the substrate. In addition, a plurality of holes or openings are formed through the thickness of the substrate. The substrate material between the openings is of a size and geometry that allows cells to adhere to the surface of the substrate material as if it were approximately a two-dimensional (2D) surface, while also allowing adequate fluid flow around the substrate material and through the openings. In some embodiments, the substrate is a polymer-based material, and can be formed as a molded polymer sheet; a polymer sheet with openings punched through the thickness; a number of filaments that are fused into a mesh-like layer; a 3D-printed substrate; or a plurality of filaments that are woven into a mesh layer. The physical structure of the matrix has a high surface-to-volume ratio for culturing anchorage dependent cells. According to various embodiments, the matrix can be arranged or packed in a bioreactor in certain ways discussed here for uniform cell seeding and growth, uniform media perfusion, and efficient cell harvest.

Embodiments of this disclosure can achieve viral vector platforms of a practical size that can produce viral genomes on the scale of greater than about 10¹⁴ viral genomes per batch, greater than about 10¹⁵ viral genomes per batch, greater than about 10¹⁶ viral genomes per batch, greater than about 10¹⁷ viral genomes per batch, or up to or greater than about g 10¹⁶ viral genomes per batch. In some embodiments, productions is about 10¹⁵ to about 10¹⁸ or more viral genomes per batch. For example, in some embodiments, the viral genome yield can be about 10¹⁵ to about 10¹⁶ viral genomes or batch, or about 10¹⁶ to about 10¹⁹ viral genomes per batch, or about 10¹⁶-10¹⁸ viral genomes per batch, or about 10¹⁷ to about 10¹⁹ viral genomes per batch, or about 10¹⁸ to about 10¹⁹ viral genomes per batch, or about 10¹⁸ or more viral genomes per batch.

In addition, the embodiments disclosed herein enable not only cell attachment and growth to a cell culture substrate, but also the viable harvest of cultured cells. The inability to harvest viable cells is a significant drawback in current platforms, and it leads to difficulty in building and sustaining a sufficient number of cells for production capacity. According to an aspect of embodiments of this disclosure, it is possible to harvest viable cells from the cell culture substrate, including between 80% to 100% viable, or about 85% to about 99% viable, or about 90% to about 99% viable. For example, of the cells that are harvested, at least 80% are viable, at least 85% are viable, at least 90% are viable, at least 91% are viable, at least 92% are viable, at least 93% are viable, at least 94% are viable, at least 95% are viable, at least 96% are viable, at least 97% are viable, at least 98% are viable, or at least 99% are viable. Cells may be released from the cell culture substrate using, for example, trypsin, TrypLE, or Accutase.

The cell culture substrate can be a woven mesh layer made of a first plurality of fibers running in a first direction and a second plurality of fibers running in a second direction. The woven fibers of the substrate form a plurality of openings, which can be defined by one or more widths or diameters. The size and shape of the openings can vary based on the type of weave (e.g., number, shape and size of filaments; angle between intersecting filaments, etc.). A woven mesh may be characterized as, on a macro-scale, a two-dimensional sheet or layer. However, a close inspection of a woven mesh reveals a three-dimensional structure due to the rising and falling of intersecting fibers of the mesh. Without wishing to be bound by theory, it is believed that the three-dimensional structure of the substrate is advantageous as it provides a large surface area for culturing adherent cells, and the structural rigidity of the mesh can provide a consistent and predictable cell culture matrix structure that enables uniform fluid flow.

In one or more embodiments, a fiber may have a diameter in a range of about 50 μm to about 1000 μm; about 100 μm to about 750 μm; about 125 μm to about 600 μm; about 150 μm to about 500 μm; about 200 μm to about 400 μm; about 200 μm to about 300 μm; or about 150 μm to about 300 μm. On a microscale level, due to the scale of the fiber compared to the cells (e.g., the fiber diameters being larger than the cells), the surface of monofilament fiber is presented as an approximation of a 2D surface for adherent cells to attach and proliferate. Fibers can be woven into a mesh with openings ranging from about 100 μm×100 μm to about 1000 μm×1000 μm. In some embodiments, the opening may have a diameter of about 50 μm to about 1000 μm; about 100 μm to about 750 μm; about 125 μm to about 600 μm; about 150 μm to about 500 μm; about 200 μm to about 400 μm; or about 200 μm to about 300 μm. These ranges of the filament diameters and opening diameters are examples of some embodiments, but are not intended to limit the possible feature sizes of the mesh according to all embodiments. The combination of fiber diameter and opening diameter is chosen to provide efficient and uniform fluid flow through the substrate when, for example, the cell culture matrix is comprises a number of adjacent mesh layers (e.g., a stack of individual layers or a rolled mesh layer).

Factors such as the fiber diameter, opening diameter, and weave type/pattern will determine the surface area available for cell attachment and growth. In addition, when the cell culture matrix includes a stack, roll, or other arrangement of overlapping substrate, the packing density of the cell culture matrix will impact the surface area of the packed bed matrix. Packing density can vary with the packing thickness of the substrate material (e.g., the space needed for a layer of the substrate). For example, if a stack of cell culture matrix has a certain height, each layer of the stack can be said to have a packing thickness determined by dividing the total height of the stack by the number of layers in the stack. The packing thickness will vary based on fiber diameter and weave, but can also vary based the alignment of adjacent layers in the stack. For instance, due to the three-dimensional nature of a woven layer, there is a certain amount of interlocking or overlapping that adjacent layers can accommodate based on their alignment with one another. In a first alignment, the adjacent layers can be tightly nestled together, but in a second alignment, the adjacent layers can have zero overlap, such as when the lower-most point of the upper layer is in direct contact with the upper-most point of the lower layer. It may be desirable for certain applications to provide a cell culture matrix with a lower density packing of layers (e.g., when higher permeability is a priority) or a higher density of packing (e.g., when maximizing substrate surface area is a priority). According to one or more embodiments, the packing thickness can be from about 50 μm to about 1000 μm; about 100 μm to about 750 μm; about 125 μm to about 600 μm; about 150 μm to about 500 μm; about 200 μm to about 400 μm; about 200 μm to about 300 μm.

The above structural factors can determine the surface area of a cell culture matrix, whether of a single layer of cell culture substrate or of a cell culture matrix having multiple layers of substrate). For example, in a particular embodiment, a single layer of woven mesh substrate having a circular shape and diameter of 6 cm can have an effective surface area of about 68 cm 2. The “effective surface area,” as used herein, is the total surface area of fibers in a portion of substrate material that is available for cell attachment and growth. Unless stated otherwise, references to “surface area” refer to this effective surface area. According to one or more embodiments, a single woven mesh substrate layer with a diameter of 6 cm may have an effective surface area of about 50 cm² to about 90 cm²; about 53 cm² to about 81 cm²; about 68 cm²; about 75 cm²; or about 81 cm². These ranges of effective surface area are provided for example only, and some embodiments may have different effective surface areas. The cell culture matrix can also be characterized in terms of porosity, as discussed in the Examples herein.

The substrate mesh can be fabricated from monofilament or multifilament fibers of polymeric materials compatible in cell culture applications, including, for example, polystyrene, polyethylene terephthalate, polycarbonate, polyvinylpyrrolidone, polybutadiene, polyvinylchloride, polyethylene oxide, polypyrroles, and polypropylene oxide. Mesh substrates may have a different patterns or weaves, including, for example knitted, warp-knitted, or woven (e.g., plain weave, twilled weave, dutch weave, five needle weave).

The surface chemistry of the mesh filaments may need to be modified to provide desired cell adhesion properties. Such modifications can be made through the chemical treatment of the polymer material of the mesh or by grafting cell adhesion molecules to the filament surface. Alternatively, meshes can be coated with thin layer of biocompatible hydrogels that demonstrate cell adherence properties, including, for example, collagen or Matrigel®. Alternatively, surfaces of filament fibers of the mesh can be rendered with cell adhesive properties through the treatment processes with various types of plasmas, process gases, and/or chemicals known in the industry. In one or more embodiments, however, the mesh is capable of providing an efficient cell growth surface without surface treatment.

The system 50 of FIG. 2 includes a bioreactor 13 housing the cell culture matrix of one or more embodiments disclosed herein. The bioreactor 13 can be fluidly connected to a media conditioning vessel 100, as described above, and the system is capable of supplying a cell culture media within the conditioning vessel 100 to the bioreactor 13. The media conditioning vessel 100 can include sensors and control components found in typical bioreactor used in the bioprocessing industry for a suspension batch, fed-batch or perfusion culture. These include but are not limited to DO oxygen sensors, pH sensors, oxygenator/gas sparging unit, temperature probes, and nutrient addition and base addition ports. A gas mixture supplied to sparging unit can be controlled by a gas flow controller for N₂, O₂, and CO₂ gasses. The media conditioning vessel 100 can also contain a pump or an impeller for media mixing. All media parameters measured by sensors listed above can be controlled by a media conditioning control unit in communication with the media conditioning vesse1100, and capable of measuring and/or adjusting the conditions of the cell culture media to the desired levels. As shown in FIG. 2, the media conditioning vessel 100is provided as a vessel that is separate from the bioreactor vessel 13. This can have advantages in terms of being able to condition the media separate from where the cells are cultured, and then supplying the conditioned media to the cell culture space. However, in some embodiments, media conditioning can be performed within the bioreactor vessel 13.

The media from the media conditioning vessel 100is delivered to the bioreactor 13 via an connector or tubing 104, which may also include an injection port for cell inoculum to seed and begin culturing of cells. The bioreactor vessel 13 may also include on or more outlets to another connector or tubing 105 through which the cell culture media exits the vessel 13. To analyze the contents of the outflow from the bioreactor 13, one or more sensors may be provided in the line. In some embodiments, the system 50 includes a flow control unit for controlling the flow into and/or out of the bioreactor 13 and/or media conditioning system 100. For example, the flow control unit may receive a signal from the one or more sensors (e.g., an 02 sensor) and, based on the signal, adjust the flow into the bioreactor 13 by sending a signal to a pump (e.g., peristaltic pump) upstream of the inlet to the bioreactor 13. Thus, based on one or a combination of factors measured by the sensors, the pump can control the flow into the bioreactor 13 to obtain the desired cell culturing conditions.

The media perfusion rate is controlled by the signal processing unit that collects and compares sensors signals from media conditioning system 100 and sensors located, for example, within or at the outlet of the bioreactor 13. Because of the pack flow nature of media perfusion through the packed bed bioreactor, nutrients, pH and oxygen gradients are developed along the packed bed. The perfusion flow rate of the bioreactor can be automatically controlled by the flow control unit operably connected to the peristaltic pump.

Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to a cell culture system comprising: a cell culture vessel enclosing an interior configured to culture cells in liquid cell culture media, the cell culture vessel comprising a bioreactor inlet for supply of cell culture media to the interior and a bioreactor outlet for removal of cell culture media from the interior; and a media conditioning system comprising an enclosure enclosing an interior space and a gas exchange system, the gas exchange system comprising a media vessel, wherein the media vessel comprises at least one media cavity configured to contain cell culture media and a gas permeable membrane separating the media cavity from the interior space.

Aspect 2 pertains to the cell culture system according to Aspect 1, wherein the enclosure is a reusable component and the gas exchange system is a single-use component.

Aspect 3 pertains to the cell culture system according to Aspect 1 or Aspect 2, wherein the enclosure is a thermal enclosure.

Aspect 4 pertains to the cell culture system according to any one of Aspects 1-3, wherein the enclosure comprises a temperature control system.

Aspect 5 pertains to the cell culture system according to any one of Aspects 1-4, wherein the media conditioning system is a closed system.

Aspect 6 pertains to the cell culture system according to any one of Aspects 1-5, wherein the gas permeable membrane is liquid impermeable.

Aspect 7 pertains to the cell culture system according to any one of Aspects 1-6, wherein the media vessel further comprises a plurality of media cavities separated by a plurality of tracheal spaces, the tracheal spaces being exposed to a gas environment in the enclosure and the gas impermeable membrane separating the tracheal spaces from the media cavities.

Aspect 8 pertains to the cell culture system of Aspect 7, wherein the media vessel further comprises an inlet manifold that connects the plurality of media cavities in fluid communication with each other.

Aspect 9 pertains to the cell culture system according to any one of Aspects 1-8, the media conditioning system further comprising a media inlet and a media outlet.

Aspect 10 pertains to the cell culture system of Aspect 9, wherein the media inlet supplies media to the at least one media cavity.

Aspect 11 pertains to the cell culture system of Aspect 9 or Aspect 10, wherein the media outlet is in fluid communication with an inlet of the cell culture vessel and is configured to output media from the at least one media cavity to the cell culture vessel.

Aspect 12 pertains to the cell culture system of any one of Aspects 9-11, wherein the media vessel further comprises an outlet manifold that is disposed between the plurality of media cavities and the media outlet, and connects the plurality of media cavities in fluid communication with the media outlet.

Aspect 13 pertains to the cell culture system according to any one of Aspects 1-12, wherein the cell culture vessel and the media conditioning system are arranged in a perfusion loop.

Aspect 14 pertains to the cell culture system according to any one of Aspects 1-13, wherein the enclosure comprises a gas inlet configured to supply gas to the interior space.

Aspect 15 pertains to the cell culture system of Aspect 14, wherein the media conditioning system comprises a gas manifold configured to supply gas from the interior space to the plurality of tracheal spaces.

Aspect 16 pertains to the cell culture system of Aspect 14 or Aspect 15, wherein the media conditioning system further comprises a gas exchanger.

Aspect 17 pertains to the cell culture system according to any one of Aspects 1-16, further comprising one or more sensors configured to measure a quantity of at least one of a gas in the enclosure and a liquid media in the media conditioning system.

Aspect 18 pertains to the cell culture system according any one of the preceding Aspects, wherein the media cavity follows a circuitous path through the media conditioning system.

Aspect 19 pertains to the cell culture system of Aspect 18, wherein the media cavity traverses a plurality of gas permeable membranes along the circuitous path, the plurality of gas permeable membranes separating media within the media cavity from a plurality of tracheal spaces.

Aspect 20 pertains to a media conditioning system for conditioning cell culture media comprising: a reusable enclosure enclosing an interior space; and a gas exchange system, the gas exchange system comprising a media vessel with at least one media cavity configured to contain cell culture media and a gas permeable membrane separating the media cavity from the interior space, wherein the gas exchange system is a single-use component.

Aspect 21 pertains to the media conditioning system according to Aspect 20, wherein the enclosure is a thermal enclosure.

Aspect 22 pertains to the media conditioning system according to Aspect 20 or Aspect 21, wherein the enclosure comprises a temperature control system.

Aspect 23 pertains to the media conditioning system according to any one of Aspects wherein the media conditioning system is a closed system.

Aspect 24 pertains to the media conditioning system according to any one of Aspects wherein the gas permeable membrane is liquid impermeable.

Aspect 25 pertains to the media conditioning system according to any one of Aspects wherein the media vessel further comprises a plurality of media cavities separated by a plurality of tracheal spaces, the tracheal spaces being exposed to a gas environment in the enclosure and the gas impermeable membrane separating the tracheal spaces from the media cavities.

Aspect 26 pertains to the media conditioning system of Aspect 25, wherein the media vessel further comprises an inlet manifold that connects the plurality of media cavities in fluid communication with each other.

Aspect 27 pertains to the media conditioning system according to any one of Aspects the media conditioning system further comprising a media inlet and a media outlet.

Aspect 28 pertains to the media conditioning system of Aspect 27, wherein the media inlet supplies media to the at least one media cavity.

### Definitions

“Wholly synthetic” or “fully synthetic” refers to a cell culture article, such as a microcarrier or surface of a culture vessel, that is composed entirely of synthetic source materials and is devoid of any animal derived or animal sourced materials. The disclosed wholly synthetic cell culture article eliminates the risk of xenogeneic contamination.

“Include,” “includes,” or like terms means encompassing but not limited to, that is, inclusive and not exclusive.

“Users” refers to those who use the systems, methods, articles, or kits disclosed herein, and include those who are culturing cells for harvesting of cells or cell products, or those who are using cells or cell products cultured and/or harvested according to embodiments herein.

“About” modifying, for example, the quantity of an ingredient in a composition, concentrations, volumes, process temperature, process time, yields, flow rates, pressures, viscosities, and like values, and ranges thereof, or a dimension of a component, and like values, and ranges thereof, employed in describing the embodiments of the disclosure, refers to variation in the numerical quantity that can occur, for example: through typical measuring and handling procedures used for preparing materials, compositions, composites, concentrates, component parts, articles of manufacture, or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture.

“Optional” or “optionally” means that the subsequently described event or circumstance can or cannot occur, and that the description includes instances where the event or circumstance occurs and instances where it does not.

The indefinite article “a” or “an” and its corresponding definite article “the” as used herein means at least one, or one or more, unless specified otherwise.

Abbreviations, which are well known to one of ordinary skill in the art, may be used (e.g., “h” or “hrs” for hour or hours, “g” or “gm” for gram(s), “mL” for milliliters, and “rt” for room temperature, “nm” for nanometers, and like abbreviations).

Specific and preferred values disclosed for components, ingredients, additives, dimensions, conditions, and like aspects, and ranges thereof, are for illustration only; they do not exclude other defined values or other values within defined ranges. The systems, kits, and methods of the disclosure can include any value or any combination of the values, specific values, more specific values, and preferred values described herein, including explicit or implicit intermediate values and ranges.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred.

It will be apparent to those skilled in the art that various modifications and variations can be made without departing from the spirit or scope of the disclosed embodiments. Since modifications, combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A cell culture system comprising:
a cell culture vessel enclosing an interior configured to culture cells in liquid cell culture media, the cell culture vessel comprising a bioreactor inlet for supply of cell culture media to the interior and a bioreactor outlet for removal of cell culture media from the interior; and
a media conditioning system comprising an enclosure enclosing an interior space and a gas exchange system, the gas exchange system comprising a media vessel,
wherein the media vessel comprises at least one media cavity configured to contain cell culture media and a gas permeable membrane separating the media cavity from the interior space.

2. The cell culture system according to claim 1, wherein the enclosure is a reusable component and the gas exchange system is a single-use component.

3. The cell culture system according to claim 1, wherein the enclosure is a thermal enclosure.

4. The cell culture system according to claim 3, wherein the enclosure comprises a temperature control system.

5. The cell culture system according to claim 1, wherein the media conditioning system is a closed system.

6. The cell culture system according to claim 1, wherein the gas permeable membrane is liquid impermeable.

7. The cell culture system according to claim 1, wherein the media vessel further comprises a plurality of media cavities separated by a plurality of tracheal spaces, the tracheal spaces being exposed to a gas environment in the enclosure and the gas impermeable membrane separating the tracheal spaces from the media cavities.

8. The cell culture system of claim 7, wherein the media vessel further comprises an inlet manifold that connects the plurality of media cavities in fluid communication with each other.

9. The cell culture system according to claim 1, the media conditioning system further comprising a media inlet and a media outlet.

10. The cell culture system of claim 9, wherein the media inlet supplies media to the at least one media cavity.

11. The cell culture system of claim 9, wherein the media outlet is in fluid communication with an inlet of the cell culture vessel and is configured to output media from the at least one media cavity to the cell culture vessel.

12. The cell culture system of claim 9, wherein the media vessel further comprises an outlet manifold that is disposed between the plurality of media cavities and the media outlet, and connects the plurality of media cavities in fluid communication with the media outlet.

13. The cell culture system according to claim 1, wherein the cell culture vessel and the media conditioning system are arranged in a perfusion loop.

14. The cell culture system according to claim 1, wherein the enclosure comprises a gas inlet configured to supply gas to the interior space.

15. The cell culture system of claim 14, wherein the media conditioning system comprises a gas manifold configured to supply gas from the interior space to the plurality of tracheal spaces.

16. The cell culture system of claim 14, wherein the media conditioning system further comprises a gas exchanger.

17. The cell culture system according to claim 1, further comprising one or more sensors configured to measure a quantity of at least one of a gas in the enclosure and a liquid media in the media conditioning system.

18. The cell culture system of claim 1, wherein the media cavity follows a circuitous path through the media conditioning system.

19. The cell culture system of claim 18, wherein the media cavity traverses a plurality of gas permeable membranes along the circuitous path, the plurality of gas permeable membranes separating media within the media cavity from a plurality of tracheal spaces.

20. A media conditioning system for conditioning cell culture media comprising:
a reusable enclosure enclosing an interior space; and
a gas exchange system, the gas exchange system comprising a media vessel with at least one media cavity configured to contain cell culture media and a gas permeable membrane separating the media cavity from the interior space,
wherein the gas exchange system is a single-use component.

21-28. (canceled)
